(19) United States
(12) Patent Application Publication
(10) Pub. No.: US 2016/0166500 A1
(43) Pub. Date: Jun. 16, 2016

(54) NOVEL HERBAL HAIR GROWTH PROMOTERS / HAIR TONIC

(71) Applicant: Kyele LLC, Diamond Bar, CA (US)

(72) Inventors: Mohana Rao Manam, Diamond Bar, CA (US); Yaqin Zhang, Changchun City (CN)

(21) Appl. No.: 14/969,571

(22) Filed: Dec. 15, 2015

Related U.S. Application Data
(60) Provisional application No. 62/091,831, filed on Dec. 15, 2014.

(51) Int. Cl. A61K 8/97; A61Q 7/00; A61K 8/73; A61K 8/04; A61K 8/36; A61K 8/49

(52) U.S. Cl. CPC A61K 8/975; A61K 8/36; A61K 8/4913; A61K 8/73; A61K 8/042; A61Q 7/00; A61K 2800/70; A61K 2800/596

(57) ABSTRACT

The invention is an invention belonging to the field of hair growth promoters and hair tonics. More, particularly, it relates to hair tonic composition for producing hair growth as well as uses related to thereto.

The novel hair tonic composition comprises plant hormones like Gibberellins, Abscisic acid, Indolebutyric acid and Lentinan with a carrier thereof.

The present invention is specifically utilized in the pharmaceutical or cosmetic fields.

### FIELD OF THE INVENTION

The invention is an invention belonging to the field of hair growth promoters and hair tonics. More, particularly, it relates to hair tonic composition for producing hair growth as well as uses related to thereto.

The novel hair tonic composition comprises plant hormones like Gibberellins, Abscisic acid, Indolebutyric acid and Lentinan with a carrier thereof.

The present invention is specifically utilized in the pharmaceutical or cosmetic fields.

### BACKGROUND OF THE INVENTION

Alopecia is the absence or slowing of hair growth in an area of the body where hair formerly grew. It may be caused by physical damage to the hair itself or to the hair follicles, but it is most often the result of changes in the natural growth cycle of hair. In some types of alopecia, the growth cycle is disrupted by some temporary situation such as a chemical imbalance or stress. However, the vast majority (95%) of cases of hair loss in both men (male pattern baldness) and women (female diffuse baldness) are genetic in origin. Below the surface of the skin is the hair root, which is enclosed within a hair follicle. At the base of the hair follicle is the dermal papilla (or papilla). The dermal papilla is fed by the bloodstream which carries nourishment to produce new hair. The dermal papilla is a structure very important to hair growth because it contains receptors for male hormones and androgens. Androgens regulate hair growth and in scalp hair androgens may cause the hair follicle to get progressively smaller and contribute to the development of alopecia.

Causes of hair loss are varied. Six major types of hair loss are namely alopecia areata, androgenetic alopecia, anagen effluvium (cancer treatment hair loss), self induced hair loss, telogen effluvium and scarring alopecia. Other types of hair loss include syphilitic alopecia (usually a manifestation or secondary syphilis), scleroderma (a disease that causes fibrosis i.e. hardening and tightening of the skin which interferes with the normal functioning of the hair follicles and growth of the hair and tinea capitis (which causes hair loss by digesting the keratin of the hair). Alopecia areata is thought to be an auto-immune disease of the hair, initially appearing as a rounded bare patch about an inch across the scalp. Alopecia areata affects both men and women equally and is often experienced first in childhood. Androgenetic alopecia accounts for 95% of all hair loss. It can affect both men and women although men experience a much greater degree of loss Natural Products in the treatment of alopecia include Aloe (Aloe barbadensis), Burdock (Arctium minus), Capsicum (Capsicum annuum L), Ginger (Zingiber officinale), Ginkgo (Ginkgo biloba), Green Tea (Camellia sinesis), Hip (Rosa canina), Lavender (Lavendula officinale), Milfoil (Achillea millefolium), Onion (Allium cepa), Pygeum (Pygeum africanum), Rattanjot (Amebia sp), Red Pepper (Capiscum annum), Rosemary (Rosamarinus officinalis), Safflower oil (Carthamus tinctorious), Saw Palmetto (Serenoa repens), Stinging Nettle (Urtica dioica) and Tea Tree Oil (Melaleuca altemifolia).

The use of plant hormones for the treatment of human tissues is limited to few applications, part of which are quite recent.

The first human body treatments were proposed in the early 1980s. In 1981, EP 0 079 074 A2 disclosed the activity of the gibberellins (used in combination with an optional proteolytic enzyme) for the prevention of hair loss and dandruff, as well as a promoter of hair sprouting and hair growth stimulators. The same inventor proposed preparations containing different species of gibberellins for dulling the hue of pigments deposited on the skin JP 59-005105 A2 in the following year.

EP 0 800 388 A1 claimed the stimulant activity of Gibberillic acid on skin cell proliferation and as a hair growth promoter. The same application proposed the use of Gibberillic acid for improving wound and ulcer healing processes.

EP 1 123 083 A1 included the Gibberillic acid between the compounds cooperating with carotenoids, polyphenols and others in the protection of skin from damages due to UV exposition.

EP 1 581 252 A1 and EP 1 587 505 BI claimed the use of Gibberellic acid as in combination with salicylic acid, optionally in combination also with jasmonic acid or zeatin, for stimulating the proliferation of fibroblasts or keratinocytes in the skin and/or stimulating the production of collagen and fibronectin by fibroblasts. The treatments are mainly proposed for the anti-ageing of skin and to promote wound healing, however, hair growth stimulation is also considered among the described effects of these treatments. However, this teaching is basically proposed as a theoretical consequence of the keratinocyte proliferation documented on cell line cultures, while no experimental data were obtained from ex-vivo or in-vivo hair follicles.

While the physiological mechanism with regard to hair health is not apparent in many respects, the following ingredients for hair tonic preparations have been heretofore used

1. Hormones such as follicular formone, adrenal cortical hormone and the like;
2. Vitamins such vitamins E, B2 and B6 and the like;

### 3. Aminoacids;

4. Crude drug extracts such as Japanese chirata extract and the like;
5. Antiphlogistics such as diphenhydramine hydrochloride, glycyrrhizin and the like;
6. Keratin-solubilizing agents such as lactic acid, resorcinol, salicylic acid and the like;
7. Scalp-stimulating agents such as benzyl nicotinate, tincture of copncum, nonylic vanilamide, tincture of cantharis and the like;
8. Germicides such as isopropyl methylphenol, alkyldiaminoethyl glycine hydrochloride solution, p-chloro-m-xylenol, quaternary ammonium salts and the like;
9. Refrigerants such as menthol and the like and
10. Humectants such as glycerin, propylene glycol, sorbitol, sodium dl-pyrrolidonecarboxylate and the like.

The above mentioned hair tonic compositions use a large amount of the certain ingredients such as germicides, scalp stimulating agents These hair tonic compositions have drawbacks with regard to safety. Hence, it is strictly required that the amount of such ingredients incorporated in to the hair tonic compositions be accurately adjusted. Further, certain ingredients for hair tonic preparations are not entirely satisfactory with respect to prevention of hair loss as well as hair sprouting and hair growth promoting effects.

Accordingly, there is a need for alternative hair tonic compositions and methods for modifying (e.g. retarding or stimulating) hair growth, which are easy to use, efficient, devoid of side effects, and which may be used alone or in combination with existing methods or agents.

### OBJECTIVES OF THE INVENTION

It is an objective of the present invention to provide novel hair tonic compositions for hair loss prevention and/or hair growth promotion comprising plant hormones.

It is an objective of the present invention to provide novel hair tonic compositions for hair loss prevention and/or hair growth promotion comprising plant hormones like Gibberellins, Abscisic acid, Indolebutyric acid and Lentinan.

It is another objective of the present invention to provide process for preparation of hair tonic compositions comprising the hair loss preventing and/or hair growth promoting active agent.

It is a further objective of the present invention to provide a method of using such novel hair loss preventing and/or hair growth promoting agent or pharmaceutical compositions comprising such agent which comprises administering to a subject in need thereof an effective amount of such agent or composition thereof.

It is a further objective of the present invention is to obviate the problems in the prior-art and to provide a novel hair tonic composition having superior hair care actions.

### SUMMARY OF THE INVENTION

In accordance with the present invention, there is provided a hair tonic composition comprising:

i) at least one active ingredient selected from Gibberellins, Abscisic acid, Indolebutyric acid and Lentinan and
ii) a carrier therefor.

According to another embodiment of the present in there is a method of promoting hair growth in a human comprising applying to scalp or hair roots of said humans a composition comprising Gibberellins, Abscisic acid, Indolebutyric acid and Lentinan as an active ingredient and a carrier therefor.

### DETAILED DESCRIPTION OF THE INVENTION

The present invention provides a novel hair tonic composition as lotion/solution/ointment/paste which has the advantage of promoting hair growth and preventing hair fall.

The inventors made investigations for a long period of time for the purpose of developing a hair restoring or growing agent capable of proliferating hair follicle cells and exhibiting the hair restoring or growing effect in the actual use or, in other words, capable of growing “lanugo hair, veilus hair or terminal hair” to such an extent that the growth can be confirmed.

The hair growth promoter of the present invention presents a combination of two or more than three plant hormones for accelerating the restoring or growing of hair.

In a specific embodiment of the present invention, there is provided a hair tonic composition comprising:

i) at least two active ingredients selected from Gibberellins, Abscisic acid, Indolebutyric acid and Lentinan and
ii) a carrier therefor.

In yet another specific embodiment of the present invention, there is provided a hair tonic composition comprising:

i) at least three active ingredients selected from Gibberellins, Abscisic acid, Indolebutyric acid and Lentinan and
ii) a carrier therefor.

In a more specific embodiment of the present invention, there is provided a hair tonic composition comprising:

i) Gibberellins, Abscisic acid, Indolebutyric acid and Lentinan and
ii) a carrier therefor.

In order to achieve the above objective the inventors employed plant hormones such as Gibberellins, Abscisic acid, Indolebutyric acid and Lentinan with excipients for promoting hair growth as well as preventing hair fall.

Gibberellins are a group of diterpenoid acids prepared by fungus, Gibberella fugikuroi, that function as plant growth regulators influencing a range of developmental processes in higher plants including stem elongation, germination, dormancy, flowering, sex expression, enzyme induction and leaf and fruit senescence. It was not known that hair fall can be reduced and hair growth could be promoted by

using it in hair compositions.

Gibberellin for use here in is present in the compositions at a level of from 0.001% to 5% by weight, preferably from 0.05% to 1.00% by weight.

Abscisic acid is an isoprenoid plant harmone produced in the roots of the plant as well as the terminal buds at the top of the plant. Abscisic acid has since been shown to regulate many aspects of plant growth and development including embryo maturation, seed dormancy, germination, cell division and elongation, floral induction, and responses to environmental stresses such as drought, salinity, cold, pathogen attack and UV radiation. It was not known that hair fall can be reduced and hair growth could be controlled by using it in hair compositions.

Abscisic acid for use here in is present in the compositions at a level of from 0.0001% to 0.5% by weight, preferably from 0.002% to 0.1% by weight.

Indolebutyric acid is a plant hormone in the auxin family. it was reported that the compound was isolated from leaves and seeds of maize and other species. It is a plant growth regulator, used to promote and accelerate root formation of plant clippings and to reduce transplant shock of nonfood ornamental nursery stock. Indolebutyric acid is also used on fruit and vegetable crops, field crops and ornamental turf to promote growth development of flowers and fruit and to increase crop yields

It was not known that hair fall can be reduced and hair growth could be controlled by using it in hair compositions. Indolebutyric acid for use here in is present in the compositions at a level of from 0.001% to 5% by weight, preferably from 0.002% to 0.1% by weight.

Lentinan is a ˜−1,3beta glucan with ˜−1,6 branching isolated from the fruit body of Shiitake tLentinula edodes) which is shown to stimulate the production of white blood cells in human cell lines.

It was not known that hair fall can be reduced and hair growth could be controlled by using it in hair compositions. Lentinan for use here in is present in the compositions at a level of from 0.0001% to 0.5% by weight, preferably from 0.002% to 0.1% by weight.

The novel hair tonic compositions of the present invention are preferably in the form of oral or topical preparations, more preferably in the form of topical preparations such as liquid, cream, gel, lotion or spray. The compositions are useful for hair loss prevention and/or hair growth promotion.

Further, the hair tonic composition of the present invention may be used as an external skin application composition such as a medicinal cosmetic composition for hair care.

The hair growth promoter in the form of novel hair tonic composition of the present invention can be in various forms such as liquid, cream, ointment, gel and aerosol forms.

They include hair cream, hair tonic, hair lotion, hair liquid, hair gel, hair spray, hair shampoo and hair rinse.

The hair tonic composition of the present invention comprises a combination of at least one of Gibberellin, Abscisic acid, Indolebutyric acid and Lentinan which are dissolved in alcohol and added into water. This lotion was applied daily into scalp and hair growth was noticed from the first month of the application

The content for the combination of the plant hormones of the present invention can be varied depending upon the form and method of application etc. The hair tonic composition of the present invention may further contain, in addition to the above essential ingredients, if necessary, an to extent not detracting from the desired effect of the present invention, various ingredients generally used in cosmetics, quasi-pharmaceuticals, pharmaceuticals etc such as oils, humectants, thickeners, anti-bacterial agents, medications, anti-oxidants, UV blockers, fragrances, colouring agents, surfactants, water and alcohols.

Further, hair tonic composition of the present invention may be administered transdermally through direct coating or spraying on the skin.

The main advantage of the present invention is that the novel hair tonic composition has both superior hair loss preventing action and hair growth promoting action in humans and other mammals.

The hair tonic action, including the hair loss preventing action and hair growth promoting action, of the hair tonic composition of the present invention was investigated by application of trichogram test on human subjects.

The following Examples will further illustrate the present invention, which by no means limit the scope of the invention.

### EXAMPLE 1

### A Hair Lotion Containing the Following Ingredients was Prepared

| S.No | Ingredients | Percent |
|---|---|---|
| 1 | Gibberellin | 0.01 |
| 2 | Abscisic acid | 0.0002 |
| 3 | Indolebutyric acid | 0.002 |
| 4 | Lentinan | 0.0001 |
| 5 | Alcohol | 5.00 |
| 6 | Purified water | Q.S |

### EXAMPLE 2

### A Hair Lotion Containing the Following Ingredients was Prepared

| S.No | Ingredients | Percent |
|---|---|---|
| 1 | Gibberellin | 0.01 |
| 2 | Abscisic acid | 0.02 |
| 3 | Indolebutyric acid | 0.2 |
| 4 | Lentinan | 0.01 |
| 5 | Alcohol | 10.00 |
| 6 | Purified water | Q.S |

Gibberellin, Abscisic acid, Indolebutyric acid and Lentinan are dissolved in alcohol and added into water.

### EXAMPLE 3

### Hair Growth Gel Composition

| S.No | Ingredients | Percent |
|---|---|---|
| 1 | Gibberellin | 0.01 |
| 2 | Abscisic acid | 0.0002 |
| 3 | Indolebutyric acid | 0.002 |
| 4 | Lentinan | 0.0001 |
| 5 | Alcohol | 5.00 |
| 6 | Dextran | 0.3 |
| 7 | Allantoin | 0.3 |
| 8 | Carboxy methyl chitosan | 2.0 |
| 9 | Water | Q.S |

Gibberellin, Abscisic acid, Indolebutyric acid and Lentinan are dissolved in alcohol. Allantoin, dextran and carboxy methyl chitosan are dissolved in water at 50° C. to which the alcoholic solution of Gibberellin, Abscisic acid, Indolebutyric acid and Lentinan is added. Cool with slow stirring till the gel sets in.

### EXAMPLE 4

### Hair Growth Gel Composition

| S.No | Ingredients | Percent |
|---|---|---|
| 1 | Gibberellin | 0.01 |
| 2 | Abscisic acid | 0.02 |
| 3 | Indolcbutyric acid | 0.2 |
| 4 | Lentinan | 0.01 |
| 5 | Alcohol | 10.00 |
| 6 | Dextran | 0.3 |
| 7 | Allantoin | 0.3 |
| 8 | Carboxy methyl chitosan | 2.0 |
| 9 | Water | Q.S |

Gibberellin, Abscisic acid, Indolebutyric acid and Lentinan are dissolved in alcohol. Allantoin, dextran and carboxy methyl chitosan are dissolved in water at 50° C. to which the alcoholic solution of Gibberellin, Abscisic acid, Indolebutyric acid and Lentinan is added. Cool with slow stirring till the gel sets in. Gel preparations of the present invention enhance longer contact and uniform application to the scalp.

We claim:

1. A hair tonic composition comprising of
i) at least one active ingredient selected from Gibberellins, Abscisic acid, Indolebutyric acid and Lentinan and
ii) a carrier therefor.

2. A hair tonic composition as claimed in claim 1 containing a combination of two or more plant hormones for accelerating the restoring or growing of hair.

3. The plant hormones according to claim 2 selected from Gibberellins, Abscisic acid, Indolebutyric acid and Lentinan with excipients for promoting hair growth as well as preventing hair fall.

4. A hair tonic composition comprising:
i) at least two active ingredients selected from Gibberellins, Abscisic acid, Indolebutyric acid and Lentinan and
ii) a carrier therefor.

5. A hair tonic composition comprising:
i) at least three active ingredients selected from Gibberellins, Abscisic acid, Indolebutyric acid and Lentinan and
ii) a carrier therefor.

6. A hair tonic composition comprising:
i) Gibberellins, Abscisic acid, Indolebutyric acid and Lentinan and
ii) a carrier therefor.

7. The novel hair tonic composition according to claim 1 in the form of oral or topical preparations.

8. The novel hair tonic composition according to claim 4 more preferably in the form of topical preparations such as liquid, tonic, shampoo, rinse, cream, gel, lotion or spray.

9. The novel hair tonic composition according to claim 4 administered transdermally through direct coating or spraying on the skin.

10. The compositions are useful for hair loss prevention and/or hair growth promotion.
